(19) United States
(12) Patent Application Publication
(10) Pub. No.: US 2008/0033300 A1
(43) Pub. Date: Feb. 7, 2008

(54) Systems and methods for monitoring temperature during electrosurgery or laser therapy

(76) Inventors: Anh Hoang, San Jose, CA (US); Ji-Dih Hu, San Jose, CA (US)

Correspondence Address: JONES DAY, 222 East 41st Street, New York, NY, 10017-6702, US

(21) Appl. No.: 11/499,530

(22) Filed: Aug. 4, 2006

(51) Int. Cl. A61B 6/00; A61B 18/18

(52) U.S. Cl. 600/474; 606/42; 606/12; 600/549

(57) ABSTRACT

Systems that measure temperatures of tissue during electrosurgery or laser therapy of the tissue or organs is provided. One system provides a pyrometer that measures infrared electromagnetic energy emitted by a surface of a tissue or organ thereby determining a sub-surface temperature of the tissue or organ. The system further has an energy generator and an ablation electrode or laser probe that delivers energy from the energy generator to a tissue or organ, responsive to a sub-surface temperature determined by the pyrometer. The pyrometer can be calibrated using a luminescent material having known optical properties as a function of temperature. The luminescent material can be positioned on the surface of the tissue or organ or inserted directly into the tissue or organ using a catheter. Methods in which the surface or sub-surface temperature of the tissue is measured during therapy are also provided.

### FIELD OF THE INVENTION

The present invention relates generally to the fields of electrosurgery and laser therapy, and more particularly to surgical and cosmetic devices and methods which employ energy (e.g., RF ablation energy, laser energy, etc.) to resect, coagulate, or ablate tissue or organs. The present invention also relates to apparatus and methods for removing tissue at a target site by a procedure that is capable of measuring tissue or organ surface temperature and/or temperatures below the surface of the tissue or organ.

### BACKGROUND OF THE INVENTION

Tissue may be destroyed, ablated, or otherwise treated using thermal energy during various therapeutic procedures. Many forms of thermal energy may be imparted to tissue, such as radio frequency electrical energy, microwave electromagnetic energy, laser energy, acoustic energy, or thermal conduction. In particular, radio frequency ablation (RFA) may be used to treat patients with tissue anomalies, such as liver anomalies and many primary cancers, such as cancers of the stomach, bowel, pancreas, kidney and lung. RFA treatment involves destroying undesirable cells by generating heat through agitation caused by the application of alternating electrical current (radio frequency energy) through the tissue. Generally, ablation therapy uses heat to kill tissue at a target site. The effective rate of tissue ablation is highly dependent on how much of the target tissue is heated to a therapeutic level. Laser-light is currently used in a large range of therapeutic and cosmetic procedures, including eye surgery, hair removal, wrinkle removal, and tattoo removal. U.S. Pat. No. 5,720,894 to Neev et al., which is incorporated herein by reference, describes biological tissue processing using an ultrashort pulse high repetition rate laser system for biological tissue processing.

Conventional electrosurgical devices and procedures, however, suffer from a number of disadvantages. For example, in certain situations, complete ablation of target tissue that is adjacent a vessel may be difficult or impossible to perform, since significant bloodflow may draw the produced heat away from the vessel wall, resulting in incomplete necrosis of the tissue surrounding the vessel. This phenomenon, which causes the tissue with greater blood flow to be heated less, and the tissue with lesser blood flow to be heated more, is known as the “heat sink” effect. It is believed that the heat sink effect is more pronounced for ablation of tissue adjacent large vessels that are more than 3 millimeters (mm) in diameter. Due to the increased vascularity of the liver, the heat sink effect may cause recurrence of liver tumors after a radio frequency ablation.

Another drawback is that conventional electrosurgery devices are not suitable for the precise removal (ablation) of tissue. Conventional electrosurgical cutting devices typically operate by creating a voltage difference between the active electrode and the target tissue, causing an electrical arc to form across the physical gap between the electrode and tissue. At the point of contact of the electric arcs with tissue, rapid tissue heating occurs due to high current density between the electrode and tissue. This high current density causes cellular fluids to rapidly vaporize into steam, thereby producing a “cutting effect” along the pathway of localized tissue heating. The tissue is parted along the pathway of vaporized cellular fluid, inducing undesirable collateral tissue damage in regions surrounding the target tissue site.

During radiofrequency energy delivery, the electrode tip temperature can be significantly lower than the tissue temperature. See, for example, 2003, Eick and Bierbaum, PACE 26, 725-730 (“Eick and Bierbaum”), which is hereby incorporated by reference in its entirety. It is believed that this is because only a thin layer of tissue adjacent to the electrode is heated directly by the electrical current (resistive heating) during radiofrequency ablation. Most of the thermal injury is thought to result from conduction of heat from the surface layer. See, for example, Nakagawa et al., 1995, Circulation 91, 2264-2273, which is hereby incorporated by reference herein in its entirety. Thus, what are needed in the art are methods for tracking the temperature below the surface of the tissue during ablation.

Eick and Bierbaum provide an experimental set up for measuring temperature in the tissue, 2 mm beneath the ablation electrode. In their setup, an ablation catheter is fixed in a holder and positioned perpendicularly to the tissue in a water basin. A force gauge measures contact force. An adjustable table allows different electrode to tissue contact settings. The catheter is connected via a connector box to the radiofrequency generator and linked to a computer for data recording. The connector box connects the thermocouple wires of a thermocouple needle with the radiofrequency generator for tissue temperature controlled radiofrequency delivery in which the temperature of the catheter tip is monitored with a thermocouple meter. In Eick and Bierbaum, the tissue is pieces of freshly excised porcine ventricle. While Eick and Bierbaum demonstrate the importance of tissue temperature controlled radiofrequency delivery in ablation procedures, the setup used by Erick and Bierbaum is wholly unsatisfactory for use in treatment of patients because it requires the injection of a thermocouple deep into the tissue being ablated.

Given the above background, what is needed in the art are improved systems and methods for tissue temperature controlled radiofrequency delivery in ablation procedures in which subsurface tissue temperatures are monitored during the ablation.

### SUMMARY OF THE INVENTION

The present in invention addresses the deficiencies found in the prior art. In one aspect of the present invention, a pyrometer measures energy in wavelengths emitted by a tissue or organ in the near infrared wavelength range in non-contact measurement mode. This near infrared energy is indicative of sub-surface temperatures of the tissue or organ. As such, the pyrometer can measure sub-surface temperatures of the tissue or organ when properly calibrated. In some embodiments, the pyrometer is calibrated using a phosphorescence probe that is either (i) on the surface of the tissue or organ or (ii) placed in a catheter that is inserted into the tissue or organ. Because the phosphorous probe is very small, the catheter can likewise be very small thus minimizing tissue or organ disturbance. In some embodiments, fluorescent material is used to measure the temperature of the tissue or organ and a pyrometer is not used.

A first aspect of the invention provides a system comprising a quantity of luminescent material, a source of transient excitation radiation, an optical fiber medium, a photodetector, a signal processor, an energy generator (e.g., an ablation energy generator), and, optionally, an ablation electrode. The luminescent material is adapted to be positioned in thermal communication with a tissue or organ and is characterized by emitting, when excited with a transient radiation source, luminescent radiation in the visible spectrum having an intensity which decreases after termination of the transient radiation. The source of transient excitation radiation is used to expose the luminescent material to an excitation radiation pulse (e.g., one microsecond or less, 100 microseconds or less, 1000 microseconds or less, one minute or less, ten minutes or less, one hour or less, a continuous pulse lasting longer than five minutes, a continuous pulse lasting longer than one hour minutes, etc.) thereby causing the luminescent material to luminesce with a decreasing intensity function having a decay time related to the temperature of the material. The optical fiber medium optically couples the transient excitation radiation with the luminescent material and collects luminescent radiation from it. The photodetector detects luminescent radiation from the luminescent material carried by the optical fiber medium as it decreases in intensity, thereby generating an electrical signal proportional thereto. The signal processor measures a decreasing characteristic of the electrical signal thereby determining a quantity that corresponds to the temperature of the luminescent material and thus also to the temperature of the tissue or organ. In some embodiments, the optional ablation electrode delivers radiation from the energy generator to the tissue or organ, responsive to a temperature of the tissue or organ determined by the signal processor. In some embodiments, the energy generator is a laser that delivers laser light to the tissue or organ, responsive to a temperature of the tissue or organ determined by the signal processor.

In some embodiments in accordance with the first aspect of the invention, the luminescent material is positioned so that it is in thermal communication with the tissue or organ surface. Thus, in such embodiments, the temperature determined by the signal processor is the tissue or organ surface temperature. In some embodiments, the system further comprises a catheter that contains the luminescent material and penetrates the tissue or organ so that the temperature of the tissue or organ determined by the signal processor is a sub-surface tissue or organ temperature. In some embodiments, the energy generator is an R-F generator, a laser, a microwave source, or an acoustic source. In some embodiments, the luminescent material has a decay constant in a range of from one microsecond to one millisecond.

The luminescent material can be any luminescent material that fluoresces at some wavelength, or range of wavelengths, as a function of temperature. For example, the luminescent material can be chromium-activated yttrium gallium garnet having the specific composition Y₃(Ga₁₋ₓCrₓ⁺³)₅O₁₂, where x in a range of 0.032 and 0.078. In another example, the luminescent material could be trivalent chromium doped yttrium aluminum garnet, having the chemical formula of Y₃(Al₁₋ₓCrₓ⁺³)₅O₁₂, where x is in a range of 0.16 and 0.060. In still another example, the luminescent material can be a trivalent chromium doped rare earth aluminum borate such as a yttrium aluminum borate, a gadolinium aluminum borate, or a lutetium aluminum borate (e.g., Gd(Al₁₋ₓCrₓ⁺³)₃(BO₃)₄ or Lu(Al₁₋ₓCrₓ⁺³)₃(BO₃)₄, where x is in a range of 0.01 to 0.04).

In some embodiments in accordance with the first aspect of the invention, the source of transient excitation radiation comprises a light emitting diode. In some embodiments, the optical fiber medium comprises a fiber or a bundle of fibers and the photodetector is a photodiode or a photo-multiplier. In some embodiments, the system further comprises a pyrometer that measures infrared electromagnetic energy emitted by a surface of the tissue or organ thereby determining a sub-surface temperature of the tissue or o. The pyrometer can be, for example, an InGaAs detector array operating in a wavelength ranges such as between 0.9 and 1.9 microns, between 1.0 and 2.2 microns, or between 1.2 and 2.6 microns. The pyrometer can measure sub-surface temperatures such as at least 1 mm below, or at least 2 mm below the surface of the tissue. In such embodiments, the pyrometer is calibrated by the temperature determined by the signal processor.

A second aspect of the present invention provides a system comprising a quantity of luminescent material, a source, a photodetector system, a signal processor, an energy generator (e.g., an ablation energy generator), and, optionally, an ablation electrode. The quantity of luminescent material is positioned in thermal communication with a tissue or organ and is characterized by emitting, when excited with a transient radiation source, luminescent radiation in a first bandwidth and a second bandwidth that are optically isolatable from each other and that each have an intensity that varies as a known function of the luminescent material. The source exposes the quantity of luminescent material to an excitation energy, thereby causing the quantity of luminescent material to luminesce. The photodetector system detects luminescent radiation from the luminescent material in the first bandwidth and the second bandwidth thereby generating a first electrical signal proportional to the first bandwidth and a second electrical signal proportional to the second bandwidth. The signal processor is responsive to the first and second electrical signals and determines the temperature of the luminescent material and thus also the temperature of the tissue or organ. In some embodiments, the optional ablation electrode delivers radiation from the ablation energy generator to the tissue or organ, responsive to the temperature of the tissue or organ determined by the signal processor. In some embodiments, the optional ablation electrode delivers radiation from the energy generator to the tissue or ogran, responsive to the temperature of the tissue or organ determined by the signal processor. In some embodiments, the energy generator is a laser that delivers laser light to the tissue or ogran, responsive to a temperature of the tissue or organ determined by the signal processor.

In some embodiments in accordance with the second aspect of the invention, the luminescent material comprises, for example, a composition (RE)₂O₂S:X where RE is lanthanum, gadolinium or yttrium, and X has a concentration of from 0.01 to 10.0 atom percent by weight and is europium, terbium, praseodymium, samarium, dysprosium, holmium, erbium, thulium, neodymium or ytterbium. In some embodiments, the luminescent material is in thermal communication with the tissue or organ surface so that the temperature determined by the signal processor is the tissue or organ surface temperature. In some embodiments, a catheter contains the luminescent material and penetrates the tissue or organ so that the temperature determined by the signal processor is a sub-surface temperature of the tissue or organ. In some embodiments, the energy generator is an R-F generator and the source is radioactive material, a source of cathode rays, or an ultraviolet electromagnetic energy source.

In some embodiments in accordance with the second aspect of the invention, the system further comprises a pyrometer that measures infrared electromagnetic energy emitted by the tissue or organ surface thereby determining a sub-surface temperature of the tissue or organ. In some embodiments, the pyrometer is an InGaAs detector array operating, for example, in the wavelength range between 0.9 and 1.9 microns, between 1.0 and 2.2 microns, or between 1.2 and 2.6 microns. In some embodiments, a sub-surface temperature determined by the pyrometer is a temperature of the tissue or organ at least 1 mm, at least 2 mm, at least 3 mm, at least 4 mm, or at least 5 mm below the tissue or organ surface.

A third aspect of the invention provides a system comprising a pyrometer, an energy generator (e.g., ablation energy generator), and, optionally, an ablation electrode. The pyrometer measures infrared electromagnetic energy emitted by a surface of a tissue or organ, thereby determining a sub-surface temperature of the tissue or organ. In some embodiments, the optional ablation electrode delivers radiation from the energy generator to the tissue or organ, responsive to the sub-surface temperature determined by the pyrometer. Accordingly, in some embodiments, the energy generator is an R-F generator. In some embodiments, the energy generator is a laser that delivers laser light to the tissue or organ, responsive to the sub-surface temperature determined by the pyrometer. In some embodiments, the pyrometer is an InGaAs detector array. In some embodiments, the sub-surface temperature determined by the pyrometer is a temperature of the tissue or organ, at least 1 mm below, least 2 mm below, at least 3 mm below, at least 4 mm below, or at least 5 mm below the tissue or organ surface.

In some embodiments in accordance with the third aspect of the invention, the system further comprises a quantity of luminescent material, a source of transient excitation radiation, an optical fiber medium, a photodetector, and a signal processor that are used to calibrate the pyrometer. The luminescent material is positioned in thermal communication with the tissue or organ and is characterized by emitting, when excited with the transient radiation source, luminescent radiation in the visible spectrum having an intensity which decreases after termination of the transient radiation. The source of transient excitation radiation exposes the luminescent material to an excitation radiation pulse, thereby causing the luminescent material to luminesce with a decreasing intensity function having a decay time that is related to the temperature of the material. The optical fiber medium optically couples the source of transient excitation radiation with the luminescent material and collects luminescent radiation from the material. The photodetector detects luminescent radiation from the material, carried by the optical fiber medium, as it decreases in intensity thereby generating an electrical signal proportional thereto. The signal processor is responsive to the electrical signal and measures a decreasing characteristic of the electrical signal to thereby determine a quantity that corresponds to the temperature of the material and thus also to the temperature of the tissue or organ. This provides a mechanism for calibrating the pyrometer. In some embodiments, the luminescent material is positioned so that it is in thermal communication with a surface of the tissue or organ so that the temperature determined by the signal processor is the tissue or organ surface temperature. In some embodiments, a catheter that contains the luminescent material penetrates the tissue or organ so that the signal processor determines a sub-surface temperature of the tissue or organ. In some embodiments, the luminescent material has a decay constant in a range of from one microsecond to one millisecond. In some embodiments, the luminescent material comprises chromium-activated yttrium gallium garnet having a specific composition Y₃(Ga₁₋ₓCrₓ⁺³)₅O₁₂, where x is in the range of 0.032 and 0.078. In some embodiments, the luminescent material comprises trivalent chromium doped yttrium aluminum garnet, having a chemical formula of Y₃(Al₁₋ₓCrₓ⁺³)₅O₁₂, where x is in the range of 0.16 and 0.060. In some embodiments, the luminescent material comprises a trivalent chromium doped rare earth aluminum borate such as yttrium aluminum borate, gadolinium aluminum borate, or lutetium aluminum borate (e.g., Gd(Al₁₋ₓCrₓ⁺³)₃(BO₃)₄ or Lu(Al₁₋ₓCrₓ⁺³)₃(BO₃)₄, where x is in the range of 0.01 to 0.04). In some embodiments, the source of transient radiation comprises a light emitting diode, the optical fiber medium comprises a fiber or a bundle of fibers, and/or the photodetector is a photodiode or a photo-multiplier.

In some embodiments in accordance with the third aspect of the invention, the system comprises a quantity of luminescent material, a source, a photodetector system, and a signal processor for calibrating the pyrometer. The luminescent material is placed in thermal communication with the tissue or organ and is characterized by emitting, when excited with a transient radiation source, luminescent radiation in a first bandwidth and a second bandwidth that are optically isolatable from each other and that each have an intensity that varies as a known function of the luminescent material. The source exposes the luminescent material to an excitation energy, thereby causing the luminescent material to luminesce. The photodetector system detects luminescent radiation from the quantity of luminescent material in the first and second bandwidths thereby respectively generating a first electrical signal proportional to the first bandwidth and a second electrical signal proportional to the second bandwidth. The signal processor, responsive to the first and second electrical signals, determines a temperature of the luminescent material and thus also a temperature of a tissue or organ. This temperature can be used to calibrate the pyrometer. The luminescent material can be, for example, a composition (RE)₂O₂S:X, where RE is lanthanum, gadolinium or yttrium and X has a concentration of from 0.01 to 10.0 atom percent by weight and is europium, terbium, praseodymium, samarium, dysprosium, holmium, erbium, thulium, neodymium or ytterbium. In some embodiments, the luminescent material is positioned so that it is in thermal communication with the tissue or organ surface so that the temperature determined by the signal processor is the tissue or organ surface temperature. In some embodiments, the luminescent material is in a catheter that penetrates the tissue or organ so that the temperature determined by the signal processor is a sub-surface tissue or organ temperature. In some embodiments, the generator is an R-F generator and the source is radioactive material, a source of cathode rays, or an ultraviolet electromagnetic energy source. In some embodiments, the generator is a laser.

A fourth aspect of the invention provides a method in which an energy source is applied to a tissue or organ site while monitoring the temperature of the tissue or organ site. In some embodiments, this energy source is applied by an ablation electrode. In some embodiments, this energy source is a laser that is applied directly to the tissue or organ site. In some embodiments, the temperature of the tissue or organ site is measured by exposing a quantity of luminescent material to the tissue or organ site such that the luminescent material is responsive to the tissue or organ site temperature. This luminescent material is characterized by emitting, when excited with a transient radiation source, luminescent radiation in the visible spectrum having an intensity which decreases after termination of the transient radiation. The luminescent material is pulsed with excitation radiation, thereby causing the material to luminesce after termination of the pulse with a decreasing intensity function having a decay time that is related to the temperature of the material. The luminescent radiation of the material is detected as it decreases in intensity thereby generating an electrical signal proportional thereto. The decreasing characteristic of the electrical signal is measured, thereby determining a quantity that corresponds to the luminescent material temperature and thus also to the tissue or organ temperature. In some embodiments, the luminescent material is positioned so that it is in thermal communication with the tissue or organ surface and the temperature monitored is therefore the surface temperature of the tissue or organ site. In some embodiments, the material is in a catheter that penetrates the tissue or organ site and the temperature monitored is a sub-surface temperature of the tissue or organ site.

In some embodiments in accordance with the fourth aspect of the invention, the temperature of the tissue or organ site that is monitored during the applying step is a sub-surface temperature (e.g. at least 1 mm, at least 2 mm, at least 3 mm, at least 4 mm, at least 5 mm below the surface of the tissue or organ) and the monitoring comprises measuring an infrared electromagnetic energy emitted by the surface of the tissue or organ site with a pyrometer. In some embodiments, the infrared electromagnetic energy is in a wavelength range between 0.9 microns and 1.9 microns. In some embodiments in accordance with the fourth aspect of the invention, the method further comprises calibrating the pyrometer. For instance, in some embodiments, the calibrating comprises exposing a quantity of luminescent material to the tissue or organ site so that the luminescent material is responsive to the tissue or organ temperature. This material is characterized by emitting, when excited with a transient radiation source, luminescent radiation in the visible spectrum having an intensity that decreases after termination of the transient radiation. The quantity of luminescent material is pulsed with an excitation radiation pulse, thereby causing the material to luminesce after termination of the pulse with a decreasing intensity function having a decay time that is related to the temperature of the material. The luminescent radiation of the material is detected as it decreases in intensity thereby generating an electrical signal proportional thereto. The decreasing characteristic of the electrical signal is measured, thereby determining a quantity that corresponds to the temperature of the luminescent material. In some embodiments, the luminescent material is positioned so that it is in thermal communication with the tissue or organ site so that the temperature of the luminescent material is a surface temperature of the tissue or organ site. In some embodiments, the luminescent material is positioned in a catheter that penetrates the tissue or organ site so that the temperature of the material is a sub-surface temperature of the tissue or organ site.

Another method of calibrating the pyrometer in accordance with the fourth aspect of the invention that uses a pyrometer comprises exposing a quantity of luminescent material to the tissue or organ site so that the material is in thermal communication with the tissue or organ. The luminescent material emits, when excited with a transient radiation source, luminescent radiation in first and second bandwidths that are optically isolatable from each other and that each have an intensity that varies as a known function of the luminescent material. A source of excitation energy is applied to the luminescent material thereby causing the material to luminesce. The luminescent radiation in the first and second bandwidths is detected thereby respectively generating a first electrical signal proportional to the first bandwidth and a second electrical signal proportional to the second bandwidth. The first and second electrical signals are evaluated to determine the temperature of the material and thus also the temperature of the tissue or organ site thereby calibrating the pyrometer. The luminescent material in such embodiments can comprises, for example, composition (RE)₂O₂S:X, where RE is lanthanum, gadolinium or yttrium and X has a concentration of from 0.01 to 10.0 atom percent by weight and is europium, terbium, praseodymium, samarium, dysprosium, holmium, erbium, thulium, neodymium or ytterbium. In some embodiments, the luminescent material is positioned so that it is in thermal communication with a surface of the tissue or organ site so that the temperature of the tissue or organ site determined in the calibration is the surface temperature of the tissue or organ site. In some embodiments, the luminescent material is in a catheter that penetrates the tissue or organ site so that the temperature of the tissue or organ determined in the calibration is a sub-surface temperature of the tissue or organ site.

In some embodiments, the tissue or organ (e.g. tissue site or organ site) is a site of a tissue disease, such as a liver anomaly, stomach cancer, bowel cancer, pancreatic cancer, kidney cancer, or lung cancer. In some embodiments, the tissue or organ is ablated during to a controlled depth by plasma-induced volumetric removal of the tissue. In some embodiments, the tissue or organ is exposed to a temperature in the range of 40° C. to 90° C. during such ablation. In some embodiments, the tissue is skin. In some embodiments, the organ is heart, bladder, lung, liver, muscle, salivary gland, colon, spleen, pancreas, gallbladder, liver, kidney, stomach, tongue, thyroid gland, gallbladder, brain, large intestine, or small intestine.

### BRIEF DESCRIPTION OF THE DRAWINGS

FIG. 1 illustrates a schematic of an in-vitro tissue temperature controlled radiofrequency delivery ablation setup in accordance with the present invention.

FIGS. 2A, 2B, and 2C illustrate three different probe configurations in accordance with the present invention.

FIG. 3 illustrates the operation of the system of FIG. 1 by showing various exemplary optical and electrical signals thereof as a function of time.

FIG. 4 is a flowchart that sets forth a sequence of operations with respect to FIG. 1.

FIGS. 5A and 5B are curves that show the characteristics of a preferred luminescent material for use with the measurement system of FIG. 1 in accordance with an embodiment of the present invention.

FIG. 6 illustrates a pyrometer used in accordance with the present invention.

Like reference numerals refer to corresponding parts throughout the several views of the drawings.

### DETAILED DESCRIPTION

Radiofrequency ablation (RFA) is used for local tissue ablation. See Decadt and Siriwardena, 2004, “Radiofrequency ablation of liver tumors: systematic review,” Lancet Oncol 5, 550-560, which is hereby incorporated by reference. In RFA, a needle electrode (e.g., 14-17.5 G) with an insulated shaft and a non-insulated distal tip is inserted into or over a lesion, often with imaging-guidance. For example, the physician may be guided in the placement of the needle by images from an imaging provided by ultrasound, a CT scanner, or magnetic resonance. In some procedures, once the needle is in place, tines are deployed from the hollow core of the needle. These tines penetrate the tissue. The patient is made into an electrical circuit by placing grounding pads in appropriate places (e.g. on the thighs or back muscles). RFA energy is then sent through the needle and tines, destroying the tissue.

Radiofrequency ablation is an attractive tool for cancer patients, especially for liver diseases and lung cancer. Since the patient's body is only penetrated with the needle in such procedures, RFA is minimally invasive. Some patients are able to return home the day of the procedure while others are observed overnight. Because the procedure is minimally invasive, patients can begin, or continue with, chemotherapy. In addition, the needle can be placed in locations unavailable to surgery, so that many tumors can be destroyed which would otherwise be inoperable. While RFA has previously been used to ablate small tumors, multiple placements of the needle can effectively ablate larger tumors as well. Even if all of the large tumor cannot be ablated, there is much to be gained from the debulking of a large tumor.

Radiofrequency ablation has also proven to be an effective and curative treatment for heart ailments including several supraventricular and ventricular tachyarrhythmias. RFA has been used for clinical applications such as osteiod osteoma, nerve ganglion ablation, and dermatological treatment. For instance, U.S. Pat. No. 7,020,528, which is hereby incorporated by reference herein in its entirety, provides a method for treatment of acne using RFA.

One drawback of RFA is the “heat-sink” effect. The heat-sink effect may occur in treated tissue adjacent to large vessels. The inflow of “cool” blood at body temperature (cool relative to the ablation temperatures) may impair the heating of the tumor cells closest to the vessels and may be the site of tumor regrowth or incomplete treatment. This heat-sink effect may also result in dimpling of the treated sphere of tissues next to the vessel. Blood vessels may also be an energy sink as blood conducts energy better than other soft tissue.

The energy at the exposed tip causes ionic agitation and frictional heat, which leads to cell death and coagulation necrosis if hot enough. If the tip is too hot, the vaporization and “charring” may cause decreased energy absorption and less treated tissue volume. In conventional systems, the impedance and temperature at the tip are monitored, and the greater output is adjusted to decrease “charring” and thus increase the volume of tissue treated. The tip temperature, which is measured and used to control the radiofrequency power output, can be significantly lower than the tissue temperature. See, for example, Kongsgaard et al., 1997, PACE 20: 1252-1260; and Haines and Verow, 1990, Circulation 82, 1034-1038, each of which is hereby incorporated by reference in its entirety. If the tip holding the needle electrode is cooled and/or tissue contact is poor, a high RF power output is required to obtain a target temperature. This might lead to overheating, a popping phenomena (Eick et al., 2000, PACE 23, 253-258), and the above-mentioned charring. Active cooling using irrigated ablation catheters has been introduced to increase lesion size. However, this further increases the difference between catheter tip temperature and tissue temperature and eliminates the possibility for feedback control of the radiofrequency power by monitoring catheter tip temperatures.

It is presently believed that some of the hottest tissue spots arise 3-5 mm below the ablation tissue surface. Thus, to prevent charring of tissue, measurement of such hot spots is desirable. With such temperature measurements in hand, the radiofrequency generator output could be adjusted to deliver the greatest level of power that does not result in an increase in electrode temperature beyond a target values such as 80° C. With this and other goals in mind, the present invention provides apparatus and methods for monitoring tissue temperature during RFA or other forms of treatement, such as laser treatment. In some embodiments, the ablation tissue or organ surface is measured using a phosphorescent material. In some embodiments the phosphorescent material is in a catheter and is in optical communication with a light tube within the catheter housing an ablation electrode. In some embodiments, such as dermatological applications, the phosphorescent material can alternatively or additionally be spread on the surface of the tissue or organ to be treated (e.g., spread on the skin). In some embodiments, the phosphorescent material is housed within a needle that punctures the tissue or organ. In such embodiments, the phosphorescent material measures the temperature of the tissue or organ at least 1 to 2 mm away from the tissue or organ surface, for example, 3-5 mm away from the tissue or organ surface. In some embodiments, in addition to or instead of the phosphorescent sensor, an optical tube in the ablation catheter is in optical communication with a pyrometer, thereby sensing infrared wavelengths emitted by the tissue. When properly calibrated, these infrared wavelengths provide an accurate measurement of ablation hot spot temperatures below the surface of the ablation tissue (e.g. 3-5 mm below the ablation tissue surface). In some embodiments, the afore-mentioned phosphorescent sensors are used to calibrate the pyrometer. In some embodiments, rather than using an ablation catheter, a laser is used.

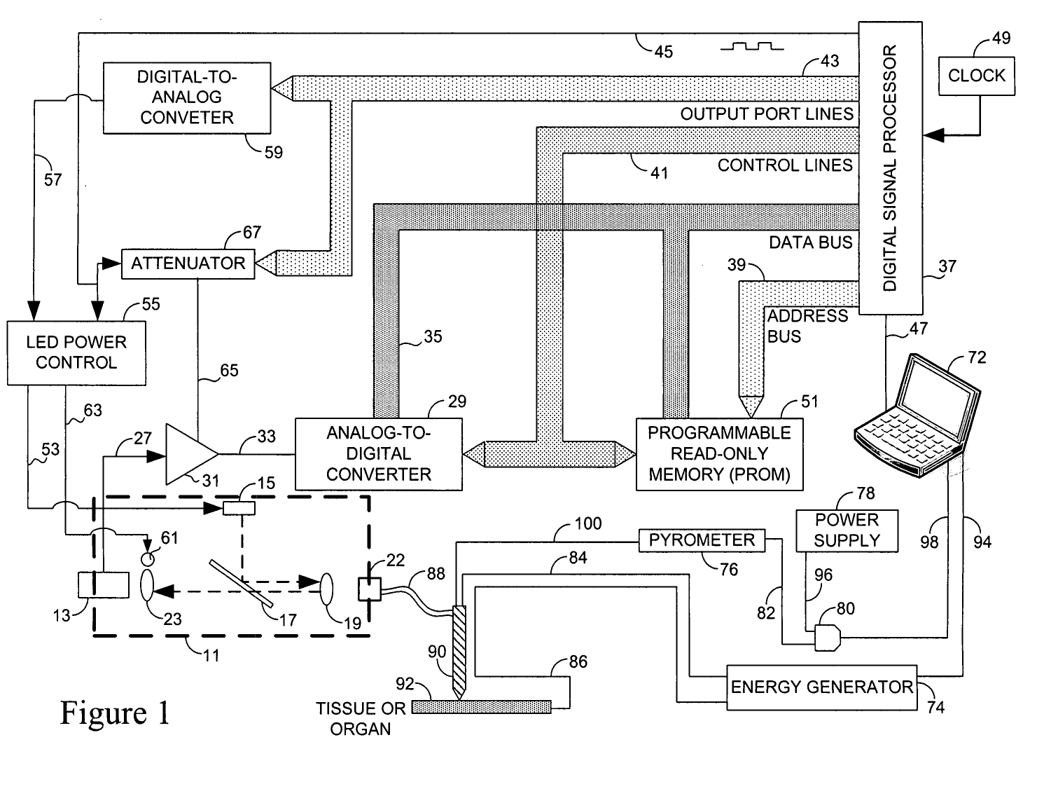

FIG. 1 illustrates an embodiment that includes (i) core components, (ii) components for measuring tissue or organ temperature using phosphorescent techniques, and (iii) components for measuring tissue or organ temperature using a pyrometer. It will be appreciated that in some embodiments, the components for measuring tissue or organ temperature using phosphorescent techniques are optional. In some alternative embodiments, the components for measuring tissue or organ temperature using a pyrometer are optional.

Referring to FIG. 1, the components for measuring issue or organ temperature using phosphorescent techniques will first be described in detail. Then, the core components will be described. Lastly the components for measuring tissue or organ temperature using a pyrometer will be described. In FIG. 1, an optical head 11 includes a photodetector 13, such as a photodiode or photo-multiplier, and a light emitting diode (LED) 15 as an excitation source. In one exemplary embodiment, LED 15 is a source of light within the red region of the visible spectrum, having a peak intensity around 650 nanometers (nm) in wavelength. Such an LED is commercially available from the Hewlett-Packard Corporation, part number HP8104, or equivalents. Its emitted light is reflected by a dichroic beam splitter 17, through lens 19, and through an optical fiber connector 22 to an end of an optical fiber transmission medium 88. Optical fiber 88 delivers the light from LED 15 to a luminescence based temperature sensor, whose luminescence is returned to optical block 11, through beam splitter 17 and lens 23 before striking detector 13. Since the excitation and luminescent wavelengths of the luminescence-based sensor are separated, dichroic beam splitter 17 is designed to reflect a majority of the excitation radiation from LED 15 while transmitting a majority of the luminescence radiation to photodiode 13. Because of the electronic signal processing utilized, as described below, no filter is required in front of photodetector 13, thus eliminating its inherent attenuation of some of the optical signal of interest.

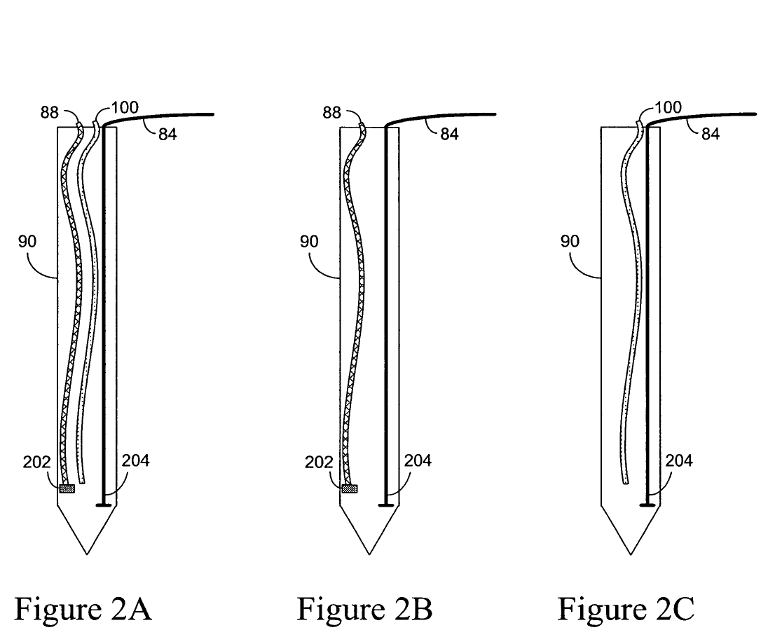

Optical fiber medium 88 can communicate with a number of forms of luminescence-based sensors. A form illustrated in FIG. 1, and in more detail in FIG. 2A, is the provision of such a sensor 202 (FIG. 2A) as part of a probe 90 carried at a free end of optical fiber medium 88. Sensor 202 is generally formed by attaching powdered luminescent material to an end of the optical fiber medium 88 with an optically clear binder. Alternatively, sensor 202 is not used and the luminescent material is attached to a tissue or organ 92 whose temperature is to be measured. A free optical fiber end of optical fiber medium 88 is then positioned to direct excitation radiation onto the luminescent material and receive the resulting luminescence back from it. The optical fiber medium end can remain spaced a distance apart from the coated surface of tissue or organ 92 in such embodiments or allowed to contact it. In the case of a large separation, auxiliary light collecting optics, such as lenses or mirrors, (not shown) may have to be used to image the fiber end onto the surface of tissue or organ 92.

Optical fiber medium 88 can be a bundle of fibers, but is preferably a single optical fiber. Indeed, one of the advantages of the system being described is that very small, single fibers may be extended for a long distance from optical block 11 to luminescence-based sensor 202 (FIG. 2A), and still provide enough signal for the measuring system of FIG. 1 to accurately extract a temperature measurement of the surface of tissue or organ 92. Any type of optical fiber may be used, rather than being restricted to the more expensive and fragile fused silica optical fibers.

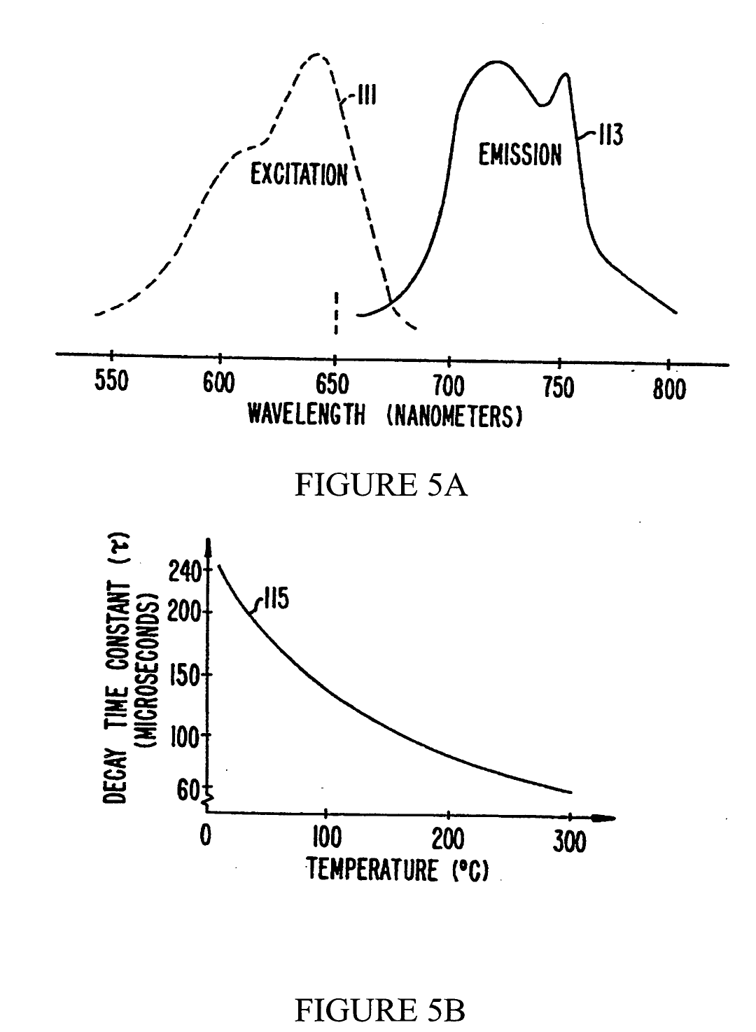

Analog signal output in line 27 from photodetector 13 is desired to be digitized by an analog-to-digital converter 29 with as few components in between as possible to reduce inherent noise generation and bandwidth restrictions. However, some analog amplification is used in some embodiments so that analog-to-digital converter 29 has enough signal to operate properly. Accordingly, in some embodiments, an input amplifier 31 is utilized, generating in line 33 an amplified version of a time varying signal output of photodetector 13. An exemplary circuit for amplifier 31 is shown in FIG. 5 of U.S. Pat. No. 5,351,268, which is hereby incorporated by reference herein in its entirety.

A digital representation of the amplified photodetector signal is outputted by analog-to-digital converter 29 onto system data bus 35. This is the data bus of signal processor 37. A representative off-the-shelf digital signal processor 37 is part number ADSP2111 of Analog Devices, Inc. This single integrated circuit chip signal processor includes connections for a separate address bus 39 and various peripheral chip control lines 41. Two output ports are provided, one of which is connected to lines 43. Various other output connections are made possible, one of which is attached to conductor 45. This particular signal processor also has connections for interfacing with host computer 72, such as through an interface bus 47. Signal processor 37 appears to host computer 72 as a peripheral device.

Alternatively, if a separate host computer interface 47 is not desired, a less expensive part number ADSP2105 signal processor of Analog Devices, Inc. can be utilized. This latter signal processor does not have provisions for a host interface but a second output port can be engineered into the system, which, unused in the system of FIG. 1, can be used to communicate with a host computer or other utilization device. In either case, a separate system clock 49 is employed. Those of skill in the art will appreciate that many other digital signal processors (DSPs) 37 can be used and all such DSPs are within the scope of the present invention.

The commercial types of signal processors identified above include a significant amount of random access memory (RAM), enough for the measurement system being described, so external RAM chips are not required. In some embodiments, a programmable read-only memory (PROM) 51 is utilized, however, and is connected to both data bus 35 and address bus 39. In some embodiments, the system operating program is contained within the PROM 51. Signal processor 37 operates, upon power-up, to load the contents of the PROM 51 into its own internal RAM. In some embodiments, signal processor 37 is an application-specific integrated circuit (ASIC) that includes sufficient RAM and logic such that PROM 51 is not necessary. In fact, in some embodiments, DSP 37, PROM 51, digital-to-analog-converter 59, and analog-to-digital converter 29 are all part of a single ASIC chip or ASIC chipset, or equivalents. However, for purposes of describing an exemplary embodiment of the present invention, these components will be considered as if they were discrete logical elements that are not part of an ASIC chip or ASIC chipset.

The system being described operates to excite sensor 202 (or in embodiments in which the fluorescent material is applied directly to the tissue, the fluorescent material by itself) to luminescence by pulsing (e.g., repetitively pulsing) the luminescent material with excitation radiation. In between pulses, characteristics of the decaying luminescence are then measured as an indication of the temperature of tissue or organ 92. Pulsing current is supplied to LED 15 through circuit 53 from power control circuits 55. Power control circuits 55 have two inputs. One is on line 45 from signal processor 37. This line contains a square wave signal that specifies the duration and frequency of the light pulses emitted by LED 15. The intensity of those pulses is controlled by an analog signal in line 57 that is the output of digital-to-analog converter 59. The level of the analog signal in line 57 is set by a digital signal in lines 43 from an output port of signal processor 37. By controlling the intensity output of the pulses of LED 15, the intensity of the resulting luminescent signal returned to photodetector 13 is controlled in order to maintain a substantially uniform signal.

An optional second LED 61 is illustrated as part of optical head 11 and driven by current in line 63 from power control circuits 55. LED 61, if used, is chosen to have a wavelength output that does not excite the luminescent sensor but to which photodiode 13 is sensitive. The purpose of LED 61 is for internal testing of the electronic system. For such testing, it is desired that there be no luminescent signal from the sensor. Power control circuits 55 periodically pulse LED 61 in accordance with the signal in line 45. In some embodiments, digital-to-analog converter 59 is conveniently chosen to be a type with an output 57 that can be driven both positively and negatively by the appropriate digital signal input in lines 43. Power control circuits 55 are then designed to utilize that feature so that a positive going signal in line 57 causes one of LEDs 15 or 61 to be pulsed with an intensity proportional to the value of that signal, while a negative-going pulse causes the other of the LEDs to be energized. Only one of LEDs 15 or 61 is energized at a single time.

Although optical head 11 is designed to minimize the amount of light output of the LED 15 that strikes photodetector 13, it is nearly impossible to prevent all such stray light from reaching the photodetector. Some excitation wavelengths are transmitted back through beam splitter 17. These wavelengths are reflected off the sensor, fiber ends and connectors, and are thus present to some degree in the signal returning to photodetector 13. Although measurement of temperature is made only during intervals between pulses when LED 15 is turned off, it is desirable to avoid driving amplifier circuits 31 to a high level during LED 15 excitation pulses. This is because of the amplifier's power rail saturation recovery time. Therefore, a signal is provided in line 65 to amplifier circuits 31 from attenuator circuit 67. Attenuator 67 receives the same pulse signal in line 45 and intensity level signal in the lines 43 as used to control LED 15. Accordingly, the timing and amount of attenuation of the signal entering amplifier circuits 31 is desirably controlled during the luminescent sensor excitation pulses. As illustrated in FIG. 1, in some embodiments, DSP 37 reports information to computer 72, such as temperature, by interface 47.

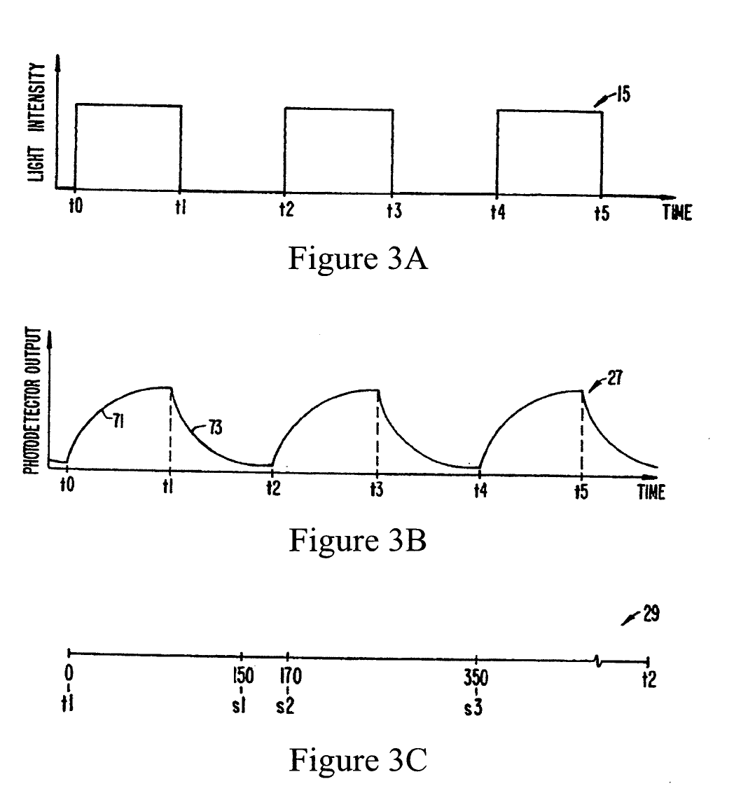

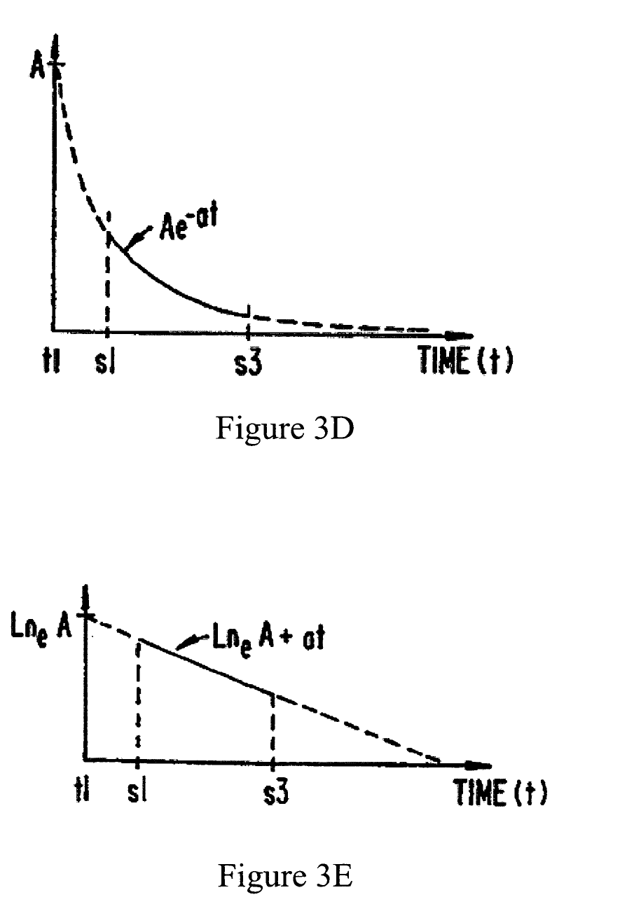

Referring to the waveforms of FIG. 3, some aspects of the operation of the system of FIG. 1 will be explained. FIG. 2A shows the excitation light pulses of the LED 15. Between times t0 and t1, LED 15 is being pulsed to direct its excitation light against the luminescent-based sensor. Between times t1 and t2, LED 15 is turned off. These pulses are periodically repeated so long as the measurement is being made. A fifty percent duty cycle of pulses is illustrated.

The luminescent signal response of the sensor to the excitation signal of FIG. 3A is shown in FIG. 3B. For the duration of an excitation pulse, the output luminescence increases in intensity, as indicated by curve 71 during the excitation pulse occurring between times t0 and t1. As soon as LED 15 is turned off, at time t1, the sensor luminescent intensity begins to drop. During the time between pulses, between times t1 and t2 of FIG. 3B, a declining signal 73 is observed by photodetector 13. The excitation pulse, between times t0 and t1, is made to be long enough to allow the sensor luminescence to substantially reach a maximum for the given excitation intensity. In the embodiment illustrated in FIG. 1, the luminescent material is preferably chosen to be of a type whose luminescence 73 decays exponentially. This facilitates measurement of changes in rate of decay that occur as a function of temperature or other parameter being measured by the sensor.

The signal of FIG. 3B is indicated to be an electrical output of photodiode 13. That output does faithfully follow the changing intensity level of the luminescence striking it if selected to have a high bandwidth. Such photodiodes are commercially available, having been developed primarily for communications applications. It is only when that signal is passed through amplifying circuits 31 that some distortion takes place because of a lower bandwidth of those circuits. It should also be noted that FIG. 3B displays only the rising luminescence signal 71 during the time that LED 15 is turned on. The effect of output radiation from the excitation LED directly striking photodiode 13, as discussed previously, has been ignored in FIG. 2B for purposes of explanation.

The portion of the signal containing the information of temperature or other parameter being measured is decaying portion 73. This is measured after each excitation pulse. A number of such measurements are then averaged to eliminate the effects of noise. The averaged decaying function is then analyzed to measure its characteristic from which the temperature or other parameter is determined.

Referring to FIG. 3C, an example of operation of the analog-to-digital converter 29 is illustrated. Its operation between intervals t1 to t2 is shown in an expanded form. Analog-to-digital converter 29 samples the decaying analog signal at a repetitive rate, beginning at time t1. But it is only during the interval between samples s1 and s3 (samples 150 and 350, respectively, in this specific example, where one sample is taken each 1.36 microseconds) that the data is utilized. This provides a fixed period of time from time t1 until sample s1 is taken for the amplifying circuits 31 to respond. Sampling is stopped at sample s3 (number 350 in this example) where the intensity of curve 73 is getting quite low.

Curve 73 as digitized after passing through the amplifier circuits 31 can be represented as follows:

Ae^{-at}+C (1)

where “C” is an offset signal generated by the photodetector and amplifying circuits 31, “e” is a natural logarithm, “t” is time, “a” is a negative reciprocal of the time constant of the exponentially decaying curve, and “A” is a beginning value of the exponentially decaying signal.

The processing accomplished by signal processor 37 first gathers a large number of sets of digital data taken from the middle of the exponentially decaying signals and combines them into a single signal, as illustrated by the solid portion of the exponential curve of FIG. 3D. In the course of this combination, a measured value of offset C is subtracted. This thus leaves a composite function as follows:

Ae^{-at} (2)

It is the quantity a, being the negative inverse of the time constant γ, that is desired to be measured. This can be accomplished by signal processor 37 by use of any number of known curve fitting techniques where parameters of an exponential are altered until that exponential matches the composite acquired signal of FIG. 3D. A least squares technique is useful for this. But it is less computational intensive, and thus faster, if a natural logarithm of the composite signal of FIG. 3D is first calculated, as shown in FIG. 3E, since it results in a straight line which is much easer to fit by standard curve fitting techniques. The log function of FIG. 3E is represented by:

LnₑA+at (3)

### Here, the desired value “a” is the slope of the straight line and more easily calculated.

Rather than calculating the quantity “a” from a composite set of digital data, it can alternatively be calculated from each set of digital data acquired for one decaying signal and then several of them averaged. This requires a higher calculating speed than is required for the averaging technique described in detail herein but may be desired in certain circumstances.

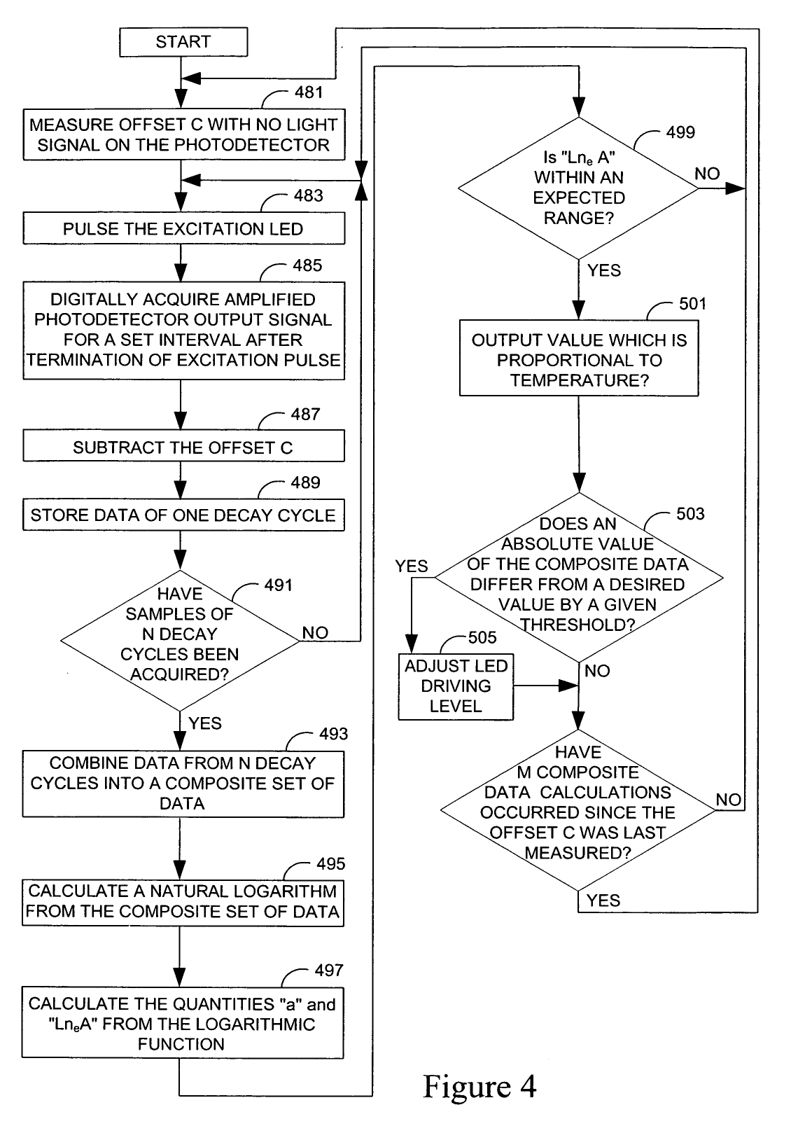

Operation of the system of FIG. 1 is more completely illustrated in the flow chart of FIG. 4. Step 481 of FIG. 4, specifying the measurement of the offset C, occurs periodically as the system is operating and is described below. Step 483 is a first step in one cycle of operation of the instrument, namely the emission of one excitation pulse of a type illustrated in FIG. 3A. A next step 485 is to digitally acquire signal 73 of FIG. 3B, as explained with respect to FIG. 3C. As explained earlier, this results in a large number of samples (e.g. 200) of the decaying signal which are acquired. A next step 487 is to subtract the offset C from those samples. This is followed by storing the corrected data samples in RAM of the signal processor 37, as indicated by a step 489.

Digital data for one cycle has then been stored. In the case where the data for a number of cycles are combined to provide a single quantity proportional to temperature, this process is repeated a number of times. Step 491 causes the process to repeat the data acquisition cycle just described until it has been done N times. After that, as indicated in step 493, data from N number of cycles is combined by averaging into a single set of data. This composite acquired signal is illustrated in FIG. 3D.

A next step, in order to simplify calculation of a time constant of this composite signal, is, as indicated at 495, to calculate a natural logarithm of the composite set of data, the results of which are illustrated in FIG. 3E. In step 497 the desired quantity, a measure of the composite acquired signal time constant proportional to temperature being measured is calculated. In addition, the logarithm of the starting point A of the composite decaying signal (Lnₑ A) is calculated for the purpose of checking the results of the A calculation.

The calculation of parameters of a curve by so-called curve-fitting techniques is well-known. For example, Press et al., Numerical Recipes—The Art of Scientific Computing, Cambridge University Press (1986), pages 498-520 of Chapter 14, which is hereby incorporated by reference, describe such techniques generally and even provide specific computer programs for carrying them out. The curve-fitting techniques initially discussed can be applied directly to the composite set of data formed by step 493, but, as previously mentioned, is a much easier and quicker calculation to do so, in step 497, on a linear set of data that results from the logarithmic calculation of step 495. Step 497 involves calculation of the two constants Lnₑ A and “a” of equation (3) given above, as illustrated in FIG. 3E.

The calculated beginning point of the decaying curve, Lnₑ A, is calculated so that it may be used in an optional step 499. This is a quantity that is not measured since no data are acquired from the decaying intensity curve immediately at the end of an excitation pulse in typical embodiments. If there are known changes in operation of the system, such as a sudden increase in the intensity of the excitation light from LED 15, the quantity “Lnₑ A” can be monitored to see if it appropriately changes in a next cycle. This quantity is independent of the temperature or other parameter being measured. But if it is detected that this quantity does not change as might be expected, such as by suddenly increasing the intensity of LED 15, then it will be known that the composite data just acquired and analyzed is likely not accurate. Such a circumstance could indicate that amplifier 31 has been driven into saturation.

In such a case, the data will be rejected and the processing commenced again with step 483. However, if no problem is detected with the data, the value of the quantity “a” is used to calculate temperature, as indicated in a step 501. The quantity a can be converted directly to temperature, for example, by use of a look-up table for the particular luminescent material being utilized as a temperature sensor.

In order to maintain the signal levels in photodetector 13 and amplifier 31 as high as possible without operating them in saturation or other non-linear operating range, the intensity of LED 15 excitation pulses is controlled as part of a feedback loop from the output. As previously mentioned, the digital-to-analog converter of FIG. 1 designates the current level that the power control circuits 55 will provide LED 15, and thus control its intensity. That intensity is set by a digital value in port lines 43. If the output signal is below a desired threshold, then the intensity of LED 15 is increased. Conversely, if the output is higher than a given threshold, then the intensity of LED 15 is decreased.

In order to determine whether the output signal level is within range or not, the absolute value of one region of the composite acquired signal curve of FIG. 3D is compared with a given threshold (Step 503). That region is preferably taken immediately after valid data samples are taken in the digitization process. Referring to FIG. 3C, the data points between samples s1 and s2 are utilized for this purpose. Thus, referring again to FIG. 4, the processing makes that comparison for that part of the composite data put together, in a step 493. If that value is outside of a specified range (503-Yes), a step 505 occurs of adjusting LED 15 intensity by changing the driving intensity number in lines 43 of FIG. 1.

A final step 507 in the processing of FIG. 5 determines whether another N cycles of decay curves are to be acquired and analyzed, beginning with step 483, or whether the offset C is again measured before doing so, by step 481. The offset C is measured periodically, every M cycles. This calculation does not have to be done very often, perhaps only every ten minutes or so, but when performed, step 481 operates in a similar mode as described when acquiring data, except that LED 15 is not pulsed. Step 483 is omitted. The offset subtraction step 487 is also omitted. Otherwise, step 481 operates similarly by acquiring digital data of the amplified output of photodetector 13 for N cycles. These data are averaged in order to calculate a new offset C that is used in subsequent data acquisition cycles. Many different luminescent materials can be used in the system described in FIG. 1. Exemplary materials are described below in the section entitled “Suitable luminescent materials that emit with a decay time as a function of temperature.”

Even with amplifier 31 being designed to have ample bandwidth, some high frequency components of an initial portion of the luminescent intensity curve are attenuated and not amplified by it. This is a reason for the delay described with respect to FIGS. 3C and 3D in acquiring data of each luminescent decay cycle. If that bandwidth can be increased without the accompanying unacceptable amplifier noise being increased, then the luminescent decay measurements can be started earlier when the intensity of the luminescent signal is desirably greater. A technique may optionally be implemented in software to accomplish this in the system being described, as illustrated in U.S. Pat. No. 5,351,268 in conjunction with FIG. 7 therein, which is hereby incorporated by reference herein in its entirety.

Details of components for measuring tissue temperature using a quantity of luminescent material have been described in conjunction with FIG. 1 and FIG. 2A. In some embodiments, these components are optional. What follows are core components of a system in accordance with one embodiment of the present invention. In some embodiments, energy generator 74 is used to generate radiofrequency energy. In some embodiments, energy generator 74 is commercially available. For example, energy generator 74 can be a Radionics RFG (Radionics Inc., Burlington, Mass.), Medtronic Atakr, or Medtronic Atakr II (Medtronic Inc., Minneapolis, USA) electrosurgical unit. In some embodiments, energy generator 74 is the drive unit of a laser. Examples of such drive units include, but are not limited to the LX2 control unit, DD2 control unit, DD control unit, and DDv control unit (Thor, Chesham, England).

In the embodiment shown in FIG. 1, energy generator 74 is linked to control computer 72 by connection 94 in order to record data from energy generator 74 and/or so that control computer 72 can control the power output of energy generator 74 as a function of either tissue 92 surface or tissue sub-temperature temperature. In some embodiments, there is no connection between energy generator 74 and computer 72. In such embodiments, temperature is reported by the fluorescent system components described above and/or the pyrometer system components described below, for example, using computer 72. In such embodiments, an operator, responsive to such temperature readings, regulates the output power of energy generator 74 and/or the position of probe 90 with respect to tissue 92.

In some embodiments, energy generator 74 delivers power to an ablation electrode 204 (FIG. 2) via coupling 84. In some embodiments, energy generator 74 delivers between 5 and 100 Watts of power. To complete the circuit, in such embodiments, a second electrode is positioned on the subject. This second electrode is connected to energy generator 74 by connection 86. In some embodiments, the second electrode is positioned on the thighs or back muscles of the subject.

In some embodiments, energy generator 74 is a laser that delivers power to a laser probe (not shown) via coupling 84. In some embodiments, energy generator 74 is a laser generator that delivers a pulse frequency between 2 Hz and 40 k Hz or a continuous laser pulse to a laser probe (not shown). Exemplary laser probes include, but are not limited to, (i) LED clusters for superficial treatments over large areas (e.g., wound healing); (ii) infra-red single laser probes for pain relieve and deep musculoskeletal disorders (e.g., joint, tendon, bone); (iii) infrared laser clusters for pain relief and deep musculoskeletal disorders over large areas; (iv) visible red single laser probes for wound healing and dermatology, and (v) visible red laser clusters for wound healing and dermatology over large areas. In embodiments where a laser probe is used, connection 86 is not required.

In typical embodiments, the subject having tissue or organ 92 to be treated is a human. However, the present application is not limited to humans. Any subject having tissue in need of radiofrequency ablation or laser therapy can benefit from the systems and methods of the present invention. Moreover, the systems and methods of the present invention have wide applicability in the research setting (e.g., to identify improved radiofrequency ablation techniques using research animals or tissue obtain from research animals).

In some embodiments, in addition to the core components, the system comprises a pyrometer 76 for measuring infrared wavelengths given off by tissue or organ 92. An example of a suitable pyrometer 76 is a PhotriX OEM pyrometer (Luxtron, Santa Clara, Calif.) using, for example, lightpipe optics. In some embodiments, pyrometer 76 is an InₓGa₁₋ₓAs pyrometer where x is a positive number less than 1 (e.g., 0.74, 0.82, etc.). In some embodiments, pyrometer 76 has a long wavelength cutoff of 1.68 μm. In some embodiments, pyrometer 76 is responsive to wavelengths in the range of about 0.9 μm to about 1.9 μm. In some embodiments, pyrometer 76 is responsive to wavelengths in the range of about 1.0 μm to about 2.2 μm. In some embodiments, pyrometer 76 is responsive to wavelengths in the range of about 1.2 μm to about 2.6 μm. Pyrometer 76 can measure temperatures over a broad range. For example, in some embodiments, pyrometer 76 can measure accurate temperature values in the temperature ranges that arise in tissue or organ 92 during treatment (e.g. 30° C. to 105° C.).

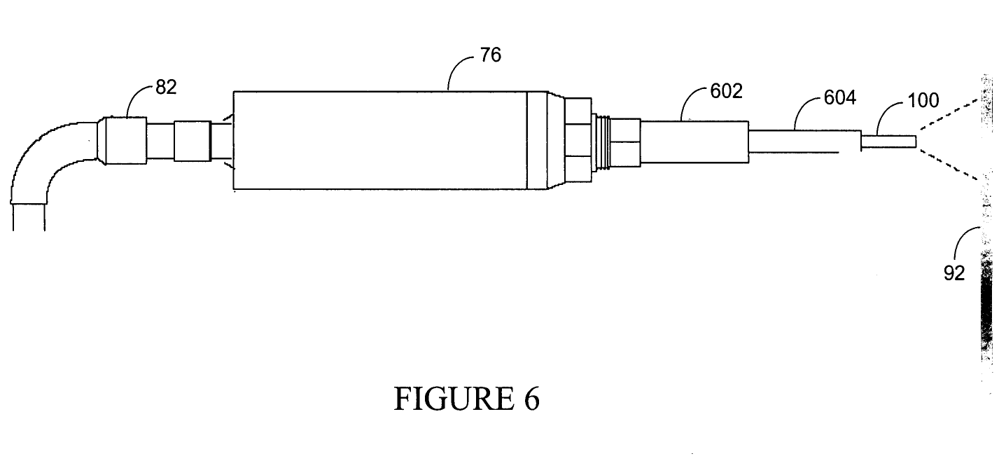

Pyrometer 76 is coupled to a site on tissue or organ 92 by connection 100. In some embodiments, connection 100 is a light pipe. FIG. 6 illustrates a combination of pyrometer 76 and connection 100, configured as a light pipe in this embodiment, in accordance with one embodiment of the present invention. The configuration illustrated in FIG. 6 includes an optional sleeve 602. Purge gas inlets can optionally be incorporated into sleeve 602. The configuration illustrated in FIG. 6 also includes an optional sheath 604. Pyrometer 76 is connected to an interface module 80 by connection 82. Interface module 80 is connected to power supply 78 by connection 96. Temperature readouts from pyrometer 76 are fed back to computer 72 by connection 98.

Now that an overview of the invention has been given, specific embodiments will be described. One embodiment of the present invention provides a laser ablation system in accordance with FIG. 1 and FIG. 2B. The laser ablation system comprises a quantity of luminescent material adapted to be positioned in thermal communication with tissue or organ 92. The luminescent material is characterized by emitting, when excited with a transient radiation source, luminescent radiation in the visible spectrum having an intensity which decreases after termination of the transient radiation. In some embodiments the luminescent material is in sensor 202 of FIG. 2B. In some embodiments, there is no sensor 202 and the luminescent material is on the surface tissue 92. The system further comprises a source of excitation radiation 22 that exposes the quantity of luminescent material to an excitation radiation pulse, thereby causing the quantity of luminescent material to luminesce with a decreasing intensity function having a decay time that is related to the temperature of the quantity of luminescent material. Optical fiber medium 88 optically couples the source of transient excitation radiation 22 with the quantity of luminescent material (e.g. 202) and collects luminescent radiation from the luminescent material. Photodetector 13 detects luminescent radiation from the quantity of luminescent material carried by optical fiber medium 88 as it decreases in intensity, thereby generating an electrical signal proportional thereto. Signal processor 37 (and/or computer 72) responsive to the electrical signal measures a decreasing characteristic of the electrical signal from detector 13, thereby determining a quantity that corresponds to the temperature of the quantity of luminescent material and thus also to the temperature of tissue 92. The system further comprises energy generator 74. In some embodiments, energy generator 74 is an ablation generator. In such embodiments, the system further comprises an ablation electrode 204 that delivers radiation from energy generator 74 to tissue or organ 92, responsive to a temperature of tissue 92 determined by signal processor 37 (and/or computer 72). In some embodiments, energy generator 74 is a laser. In some such embodiments, the system further comprises a laser probe (not shown) that delivers laser light from energy generator 74 to tissue or organ 92, responsive to a temperature of tissue 92 determined by signal processor 37 (and/or computer 72).

Now referring to FIG. 2A, in some embodiments, the system described in conjunction with FIG. 1 and FIG. 2B further comprises a pyrometer 76 that measures infrared electromagnetic energy emitted by a surface of tissue or organ 92 thereby determining a sub-surface temperature of the tissue. In some embodiments, the pyrometer is an InGaAs detector array that operates in an infrared wavelength range such as between 0.9 microns and 1.9 microns, between 1.0 microns and 2.2 microns, or between 1.2 microns and 2.6 microns. Pyrometer 100 includes a light pipe 100 that extends into probe 100 and senses infrared radiation emitted by tissue or organ 92. In some embodiments, pyrometer 76 can measure a sub-surface temperature at least 1 mm below, at least about 2 mm below, at least about 3 mm below, or between 2 and 5 mm below the surface of tissue or organ 92 during an RFA procedure. In embodiments that include both the luminescent radiation and pyrometer 76, the luminescent radiation can be used to calibrate pyrometer 76.

Now referring to FIG. 1 and FIG. 2C, one aspect of the invention provides a system comprising a pyrometer 76 that measures infrared electromagnetic energy emitted by a surface of tissue or organ 92, thereby determining a sub-surface temperature of the tissue or organ. The system in accordance with this aspect of the invention further comprises an energy generator 74 and, optionally, an ablation electrode 204 that delivers radiation from energy generator 74 to tissue or organ 92, responsive to a sub-surface temperature determined by pyrometer 76. In such embodiments, pyrometer 76 is equipped with a light pipe 100 or comparable device to receive infrared electromagnetic energy from the surface of the tissue or organ. In some embodiments in accordance with any one of FIGS. 2A, 2B, and 2C, there is a laser probe that delivers laser light to tissue or organ 92 rather than ablation electrode 204.

In some embodiments, rather than measuring time decay, the system illustrated in FIG. 1 measures light from luminescent materials that emit in two different wavelength ranges. Exemplary phosphorescent materials useful for such purpose and the circuitry that can measure such different wavelength ranges is disclosed, for example, in U.S. Pat. No. 4,560,286, which is hereby incorporated by reference herein in its entirety.

Energy generators 74 that are RF-generators have been described. However, the present invention is not limited to RF-generators. Indeed, any energy source suitable for electrosurgery and laser therapy, and more particularly for use with surgical and cosmetic methods that use energy to resect, coagulate, or ablate tissue or organs, is within the scope of the present invention and may serve the purpose of energy generator 74 in FIG. 1. Representative energy generators include, but are not limited to, lasers, radio frequencies, microwaves, and light.

In some embodiments, tissue or organ 92 is any association of cells of a multicellular organism, with a common embryological origin or pathway and similar structure and function. Often, cells of a tissue or organ 92 are contiguous at cell membranes. In the present invention, tissues are generally solid rather than liquid (e.g. blood). However, in some embodiments, the tissue is liquid. Cells in tissue 92 may be all of one type (a simple tissue, e.g. squamous epithelium) or of more than one type (a mixed tissue, e.g., connective tissue). Tissues aggregate to form organs. Thus, in some embodiments, tissue 92 is in fact an organ. An organ is a functional and anatomical unit of most multicellular organisms, consisting of at least two tissue types (often several) integrated in such a way as to perform one or more recognizable functions in the organism. Examples in animals include liver, kidney and skin. Additional examples of tissue or organ 92 include, but are not limited to heart, bladder, lung, liver, muscle, salivary gland, colon, spleen, pancreas, gallbladder, liver, kidney, stomach, tongue, thyroid gland, gallbladder, brain, large intestine, and small intestine.

Suitable luminescent materials that emit in two different wavelength ranges. The fundamental characteristics of one form of phosphor material for use in the present invention is that when properly excited it emits radiation in at least two different wavelength ranges that are optically isolatable from one another, and further that the intensity variations of the radiation within each of these at least two wavelength ranges as a function of the phosphor temperature are known and different from one another. A phosphor material is preferred that is further characterized by its radiation emission in each of these at least two wavelength bands being sharp lines that rise from substantially zero emission on either side to a maximum line intensity, all in less than 100 angstroms. The lines are easy to isolate and have their own defined bandwidth. But mixtures of broadband emitters, such as of more conventional non-rare earth phosphors, are also usable so long as two different wavelength ranges of emission of the two materials can be separated sufficiently from one another so that an intensity ratio can be taken, and as long as the temperature dependences for thermal quenching are sufficiently different for the two phosphors.

For a practical temperature measuring device, the phosphor material selected should also emit radiation in the visible or near visible region of the spectrum since this is the easiest radiation to detect with available detectors, and since radiation in this region is readily transmitted by glass or quartz windows, fibers, lenses, etc. It is also desirable that the phosphor material selected be an efficient emitter of such radiation in response to some useful and practical form of excitation of the phosphor material. The particular phosphor material or mixture of phosphor materials is also desirably chosen so that the relative change of intensity of emission of radiation within the two wavelength ranges is a maximum within the temperature range to be measured. The phosphor material should also be durable, stable and be capable of reproducing essentially the same results from batch to batch. In the case of fiber optic transmission of the phosphor emission, as described in specific embodiments hereinafter, a sharp line emitting phosphor is desirably selected with the lines having wavelengths near one another so that any wavelength dependent attenuation of the fiber optic will not significantly affect the measured results at a position remote from the phosphor, thereby eliminating or reducing the necessity for intensity compensation that might be necessary if fibers of varying lengths were used.

The composition of a phosphor material capable of providing the characteristics outlined above may be represented very generally by the generic chemical compound description AₓB_{y}C_{z}, where A represents one or more cations, B represents one or more anions, A and B together form an appropriate non-metallic host compound, and C represents one or more activator elements that are compatible with the host material. Here, x and y are small integers and z is typically in the range of a few hundredths or less.

There is a large number of known existing phosphor compounds from which those satisfying the fundamental characteristic discussed above may be selected. A preferred group of elements from which the activator element C is chosen is any of the rare earth ions having an unfilled f-electron shell, all of which have sharp isolatable fluorescent emission lines of 10 angstroms bandwidth or less. Certain of these rare earth ions having comparatively strong visible or near visible emission are preferred for convenience of detecting, and they are typically in the trivalent form: praseodymium (Pr), samarium (Sm), europium (Eu), terbium (Th), dysprosium (Dy), holmium (Ho), erbium (Er) and thulium (Tm). Other activators such as neodymium (Nd) and ytterbium (Yb) might also be useful if infra-red sensitive detectors are used. Other non-rare earth activators having a characteristic of sharp line emission which might be potentially useful in the present invention would include uranium (U) and chromium (Cr³⁺). The activator ion is combined with a compatible host material with a concentration of something less than 10 atom percent relative to the other cations present, and more usually less than 1 atom percent, depending on the particular activator elements and host compounds chosen.

A specific class of compositions that might be included in the phosphor layer is a rare earth phosphor having the composition (RE)₂O₂S:X, wherein RE is one element selected from the group consisting of lanthanum (La), gadolinium (Gd) and yttrium (Y), and X is one doping element selected from the group of rare earth elements listed in the preceding paragraph having a concentration in the range of 0.01 to 10.0 atom percent as a substitute for the RE element. A more usual portion of that concentration range will be a few atom percent and in some cases less than 0.1 atom percent. The concentration is selected for the particular emission characteristics desired for a given application. Such a phosphor compound may be suspended in an organic binder, a silicone resin binder or a potassium silicate binder. Certain of these binders may be the vehicle for a paint which can be maintained in a liquid state until thinly spread over a surface whose temperature is to be measured where it will dry and thus hold the phosphor on the surface in heat conductive contact with it.

A specific example of such a material is europium-doped lanthanum oxysulfide (La₂O₂S:Eu) where europium is present in the range of a few atom percent down to 0.01 atom percent as a substitute for lanthanum. More information on phosphors suitable with this embodiment of the present invention is provided in U.S. Pat. No. 4,560,286, which is hereby incorporated by reference in its entirety.

Suitable luminescent materials that emit with a decay time as a function of temperature. Many specific luminescent material compositions can be utilized for the sensor in the system being described in accordance with this embodiment of the invention. The material must be stable over time and up to temperatures in excess of those to be measured. The chosen luminescent composition also needs to be strongly absorptive of the radiation output of available LEDs, and emit luminescent radiation in wavelength ranges to which available high bandwidth (fast responding) photodetectors are available. The luminescent sensor composition chosen should also be easily reproducible in order to reduce variations in characteristics between different sensors. The luminescent material preferably has a decay time constant in a range of from one microsecond to one millisecond. Within this range, the requirements placed on the electronic system are not too severe, yet repeated measurements can still be made with a sufficiently high rate.

An exemplary luminescent material used in some embodiments is a chromium-activated yttrium gallium garnet having a specific composition Y₃(Ga₁₋ₓCrₓ⁺³)₅O₁₂, where X lies substantially within a range of 0.032 to 0.078, representing a concentration of the trivalent chromium activator of from 2.0 to 4.5 percent by weight. FIG. 5A shows this material's absorption spectra 111, in the red, and its emission spectrum 113, in the near-infrared. The emission spectrum 113 corresponds with the spectral sensitivity of available fast silicon photodiodes that can serve as the photodetector for the emission. The decay time constant τ of the emission of this luminescent material is the function of its temperature as illustrated in FIG. 5B. Its τ is typically within a range of 190 to 250 microseconds when the luminescent material is at room temperature (approximately 20° C.). As can be seen from FIG. 5B, the decay time constant varies from about 280 microseconds at 0° C. to around 60 microseconds at 300° C. The sensitivity of the measurement over this temperature range is good, another requirement of a satisfactory luminescent material sensor. The curves of FIG. 5 show the characteristics of the trivalent chromium activated yttrium gallium garnet luminescent material with the x of the chemical formula given above being substantially 0.47, representing a concentration of about three percent by weight of trivalent chromium.

An advantage of the opto-electronic system described above is that it can work with a luminescent material having a short decay time. The preferred material whose characteristics are illustrated in FIG. 5B have decay time constants significantly less than one millisecond for a full temperature range of interest, such as −190° C. to +400° C. Luminescent materials with time constants to be measured that are less than one or two milliseconds create greater demands on the opto-electronic measurement system utilizing them. However, when such short decay times can be handled, as they are with the system described here, there is an advantage in that a large number of decay time measurements may be taken in a very short period of time. In some embodiments, digital samples of the decaying signal are taken one microsecond apart. In some embodiments, 100 decay time cycles or more are measured and averaged together to form a single average decay time constant from which temperature or other parameters can be determined. Thus, a time constant is calculated about once each second, that calculation resulting from an average of about 100 individual decay time measurements. Providing a new measurement every second provides a real time monitoring of temperature or other parameters.

Another specific luminescent material that is suitable is a trivalent chromium doped yttrium aluminum garnet, having a chemical formula of Y₃(Al₁₋ₓCrₓ⁺³)₅O₁₂, where x lies within a range of 0.16 to 0.060, representing a concentration of trivalent chromium dopant of from one to four percent by weight. This material has a luminescence that is less bright than that of Y₃(Ga₁₋ₓCrₓ⁺³)₅O₁₂, and has a much longer time constant. Its excitation, absorption and luminescent spectra are, however, quite similar.

Trivalent chromium doped rare earth aluminum borate materials can also be used. Found to have excitation, absorption and luminescent emission spectra similar to the preferred material described above, and with the same or greater luminescent brightness, and with a shorter decay time constant, are certain yttrium aluminum, gadolinium aluminum and lutetium aluminum borates. Examples are chemical compositions Gd(Al₁₋ₓCrₓ⁺³)₃(BO₃)₄ and Lu(Al₁₋ₓCrₓ⁺³)₃(BO₃)₄, where x is generally in the range of from 0.01 to 0.04. More information on phosphors suitable with this embodiment of the present invention is provided in U.S. Pat. No. 5,351,268, which is hereby incorporated by reference in its entirety.

In some embodiments, energy generator 74 generates a pulsed laser. In other embodiments, energy generator 74 generates a laser beam that irradiates continuous energy. In some embodiments, a pulsed laser used in the present invention has a pulse frequency in the range of 0.1 kilohertz (kHz) to 1000 kHz. In some embodiments, a pulsed laser has a pulse duration in the range of 10 nanoseconds to 3.0×10⁷ nanoseconds. In some embodiments, energy generator 74 and an associated laser probe is a gas, liquid, or solid laser. Exemplary gas lasers include, but are not limited to, He—Ne, He—Cd, Cu vapor, Ag vapor, HeAg, NeCu, CO₂, N₂, HF-DF, far infrared, F₂, XeF, XeCl, ArF, KrCl, or KrF laser. Exemplary liquid lasers include dye lasers. Exemplary solid lasers include, but are not limited to, ruby, Nd:YAG, Nd:glass, color center, alexandrite, Ti:sapphire, Yb:KGW, Yb:KYW, Yb:SYS, Yb:BOYS, Yb:CaF₂, semiconductor, glass or optical fiber hosted lasers, vertical cavity surface-emitting laser (VCSEL), or laser diode laser. In some embodiments, a laser beam is generated by an x-ray, infrared, ultraviolet, or free electron transfer laser. In some embodiments, a laser beam has a wavelength in the range of 10 nanometers to 1×10⁶ nanometers. In some embodiments, a dose of radiant energy containing radiant energy in a range from 0.01 Joules per square centimeters (J/cm²) to 50.0 J/cm² is delivered to a designated area by a laser beam.

### CONCLUSION AND REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed:

1. A system comprising:
a quantity of luminescent material adapted to be positioned in thermal communication with a tissue or organ, said quantity of luminescent material being characterized by emitting, when excited with a transient radiation source, luminescent radiation in the visible spectrum having an intensity which decreases after termination of the transient radiation;
a source of transient excitation radiation that exposes said quantity of luminescent material to an excitation radiation pulse, thereby causing said quantity of luminescent material to luminesce with a decreasing intensity function having a decay time that is related to the temperature of the quantity of luminescent material;
an optical fiber medium that optically couples said source of transient excitation radiation with said quantity of luminescent material and collects luminescent radiation from the quantity of luminescent material;
a photodetector that detects luminescent radiation from the quantity of luminescent material carried by said optical fiber medium as it decreases in intensity, thereby generating an electrical signal proportional thereto;
a signal processor responsive to said electrical signal that measures a decreasing characteristic of said electrical signal, thereby determining a quantity that corresponds to the temperature of the quantity of luminescent material and thus also to the temperature of a tissue;
an energy generator; and
an ablation electrode or laser probe that delivers energy from the energy generator to a tissue, responsive to a temperature of a tissue determined by said signal processor.

2. The system of claim 1, wherein said quantity of luminescent material is positioned so that it is in thermal communication with a surface of a tissue or an organ so that a temperature determined by said signal processor is a tissue or organ surface temperature.

3. The system of claim 1, further comprising a catheter that contains said quantity of luminescent material and penetrates a surface of a tissue or an organ so that a temperature of a tissue or an organ determined by said signal processor is a sub-surface tissue or organ temperature.

4. The system of claim 1, wherein said energy generator is an R-F generator, a laser, a microwave source, or an acoustic source.

5. The system of claim 1, wherein said quantity of luminescent material has a decay constant in a range of from one microsecond to one millisecond.

6. The system of claim 1, wherein said quantity of luminescent material comprises chromium-activated yttrium gallium garnet having a specific composition Y₃(Ga₁₋ₓCrₓ⁺³)₅O₁₂, where x in a range of 0.032 and 0.078.

7. The system of claim 1, wherein said quantity of luminescent material comprises trivalent chromium doped yttrium aluminum garnet, having a chemical formula of Y₃(Al₁₋ₓCrₓ⁺³)₅O₁₂, where x is in a range of 0.16 and 0.060.

8. The system of claim 1, wherein said quantity of luminescent material comprises a trivalent chromium doped rare earth aluminum borate.

9. The system of claim 8, wherein said trivalent chromium doped rare earth aluminum borate comprises a yttrium aluminum borate, a gadolinium aluminum borate, or a lutetium aluminum borate.

10. The system of claim 8, wherein said trivalent chromium doped rare earth aluminum borate comprises Gd(Al₁₋ₓCrₓ⁺³)₃(BO₃)₄ or Lu(Al₁₋ₓCrₓ⁺³)₃(BO₃)₄, where x is in a range of 0.01 to 0.04.

11. The system of claim 1, wherein said source of transient excitation radiation comprises a light emitting diode.

12. The system of claim 1, wherein said optical fiber medium comprises a fiber or a bundle of fibers.

13. The system of claim 1, wherein said photodetector is a photodiode or a photo-multiplier.

14. The system of claim 1, further comprising:
a pyrometer that measures infrared electromagnetic energy emitted by a surface of a tissue or an organ thereby determining a sub-surface temperature of said tissue or said organ.

15. The system of claim 14, wherein said pyrometer is an InGaAs detector array.

16. The system of claim 14, wherein said pyrometer operates in a wavelength range between 0.9 microns and 1.9 microns.

17. The system of claim 14, wherein said pyrometer operates in a wavelength range between 1.0 microns and 2.2 microns.

18. The system of claim 14, wherein said pyrometer operates in a wavelength range between 1.2 microns and 2.6 microns.

19. The system of claim 14, wherein a sub-surface temperature determined by said pyrometer is a temperature of a tissue or an organ at least 1 mm below a surface of said tissue or said organ.

20. The system of claim 14, wherein a sub-surface temperature determined by said pyrometer is a temperature of a tissue or an organ at least 2 mm below a surface of said tissue or said organ.

21. A system comprising:
a quantity of luminescent material adapted to be positioned in thermal communication with a tissue or an organ, said quantity of luminescent material being characterized by emitting, when excited with a transient radiation source, luminescent radiation in a first bandwidth and a second bandwidth that are optically isolatable from each other and that each have an intensity that varies as a known function of the luminescent material;
a source that exposes said quantity of luminescent material to an excitation energy, thereby causing said quantity of luminescent material to luminesce;
a photodetector system that detects luminescent radiation from the quantity of luminescent material in the first bandwidth and the second bandwidth thereby generating a first electrical signal proportional to the first bandwidth and a second electrical signal proportional to the second bandwidth;
a signal processor, responsive to the first electrical signal and the second electrical signal, that determines the temperature of the luminescent material and thus also to the temperature of a tissue or an organ;
an energy generator; and
an ablation electrode or laser probe that delivers energy from the energy generator to a tissue or an organ, responsive to a temperature determined by said signal processor.

22. The system of claim 21, wherein said quantity of luminescent material comprises a composition (RE)₂O₂S:X, wherein
RE is an element selected from the group consisting of lanthanum, gadolinium and yttrium; and
X has a concentration of from 0.01 to 10.0 atom percent by weight and is selected from the group consisting of europium, terbium, praseodymium, samarium, dysprosium, holmium, erbium, thulium, neodymium and ytterbium.

23. The system of claim 21, wherein said quantity of luminescent material is positioned so that it is in thermal communication with a surface of a tissue or an organ so that a temperature determined by said signal processor is a surface temperature of a tissue or an organ.

24. The system of claim 21, further comprising a catheter that contains said quantity of luminescent material and penetrates a surface of a tissue or an organ so that a temperature determined by said signal processor is a tissue or an organ sub-surface temperature.

25. The system of claim 21, wherein said energy generator is an R-F generator, a laser generator, a microwave source, or an acoustic source.

26. The system of claim 21, wherein said source is radioactive material, a source of cathode rays, or an ultraviolet electromagnetic energy source.

27. The system of claim 21, further comprising:
a pyrometer that measures infrared electromagnetic energy emitted by a surface of a tissue or a surface of an organ thereby determining a sub-surface temperature of a tissue or an organ.

28. The system of claim 27, wherein said pyrometer is an InGaAs detector array.

29. The system of claim 27, wherein said pyrometer operates in a wavelength range between 0.9 microns and 1.9 microns.

30. The system of claim 27, wherein said pyrometer operates in a wavelength range between 1.0 microns and 2.2 microns.

31. The system of claim 27, wherein said pyrometer operates in a wavelength range between 1.2 microns and 2.6 microns.

32. The system of claim 27, wherein a sub-surface temperature determined by said pyrometer is a temperature of a tissue or an organ at least 1 mm below a surface of a tissue or an organ.

33. The system of claim 27, wherein a sub-surface temperature determined by said pyrometer is a temperature of a tissue or an organ at least 2 mm below a surface of a tissue or an organ.

34. A system comprising:
a pyrometer that measures infrared electromagnetic energy emitted by a surface of a tissue or a surface of an organ, thereby determining a sub-surface temperature of a tissue or an organ;
an energy generator; and
an electrode or a laser probe that delivers energy from the energy generator to a tissue, responsive to a sub-surface temperature determined by said pyrometer.

35. The system of claim 34, wherein said energy generator is an R-F generator, a laser generator, a microwave source, or an acoustic source.

36. The system of claim 34, wherein said pyrometer is an InGaAs detector array.

37. The system of claim 34, wherein said pyrometer operates in a wavelength range between 0.9 microns and 1.9 microns.

38. The system of claim 34, wherein said pyrometer operates in a wavelength range between 1.0 microns and 2.2 microns.

39. The system of claim 34, wherein said pyrometer operates in a wavelength range between 1.2 microns and 2.6 microns.

40. The system of claim 34, wherein a sub-surface temperature determined by said pyrometer is a temperature of a tissue or an organ at least 1 mm below a surface of a tissue or an organ.

41. The system of claim 34, wherein a sub-surface temperature determined by said pyrometer is a temperature of a tissue or an organ at least 2 mm below a surface of a tissue or an organ.

42. The system of claim 34, further comprising:
a quantity of luminescent material adapted to be positioned in thermal communication with a tissue or an organ, said quantity of luminescent material being characterized by emitting, when excited with a transient radiation source, luminescent radiation in the visible spectrum having an intensity which decreases after termination of the transient radiation;
a source of transient excitation radiation that exposes said quantity of luminescent material to an excitation radiation pulse, thereby causing said quantity of luminescent material to luminesce with a decreasing intensity function having a decay time that is related to a temperature of the quantity of luminescent material;
an optical fiber medium that optically couples said source of transient excitation radiation with said quantity of luminescent material and collects luminescent radiation from the quantity of luminescent material;
a photodetector that detects luminescent radiation from the quantity of luminescent material carried by said optical fiber medium as it decreases in intensity thereby generating an electrical signal proportional thereto; and
a signal processor responsive to the electrical signal generated by the photodetector that measures a decreasing characteristic of the electrical signal and determines a quantity that corresponds to the temperature of the quantity of luminescent material and thus also to the temperature of a tissue or an organ, thereby calibrating said pyrometer.

43. The system of claim 42, wherein said quantity of luminescent material is positioned so that it is in thermal communication with a surface of a tissue or an organ so that a temperature of a tissue or an organ determined by said signal processor is a surface temperature of a tissue or an organ.

44. The system of claim 42, further comprising a catheter that contains said quantity of luminescent material and penetrates a surface of a tissue or an organ so that a temperature of a tissue or an organ determined by said signal processor is a sub-surface temperature of a tissue or an organ.

45. The system of claim 42, wherein said quantity of luminescent material has a decay constant in a range of from one microsecond to one millisecond.

46. The system of claim 42, wherein said quantity of luminescent material comprises chromium-activated yttrium gallium garnet having a specific composition Y₃(Ga₁₋ₓCrₓ⁺3)₅O₁₂, where x is in the range of 0.032 and 0.078.

47. The system of claim 42, wherein said quantity of luminescent material comprises trivalent chromium doped yttrium aluminum garnet, having a chemical formula of Y₃(Al₁₋ₓCrₓ⁺³)₅O₁₂, where x is in the range of 0.16 and 0.060.

48. The system of claim 42, wherein said quantity of luminescent material comprises a trivalent chromium doped rare earth aluminum borate.

49. The system of claim 48, wherein said trivalent chromium doped rare earth aluminum borate comprises a yttrium aluminum borate, a gadolinium aluminum borate, or a lutetium aluminum borate.

50. The system of claim 48, wherein said trivalent chromium doped rare earth aluminum borate comprises Gd(Al₁₋ₓCrₓ⁺³)₃(BO₃)₄ or Lu(Al₁₋ₓCrₓ⁺³)₃(BO₃)₄, where x is in the range of 0.01 to 0.04.

51. The system of claim 42, wherein said source of transient radiation comprises a light emitting diode.

52. The system of claim 42, wherein said optical fiber medium comprises a fiber or a bundle of fibers.

53. The system of claim 42, wherein said photodetector is a photodiode or a photo-multiplier.

54. The system of claim 34, further comprising:
a quantity of luminescent material adapted to be positioned in thermal communication with a tissue or an organ, said quantity of luminescent material being characterized by emitting, when excited with a transient radiation source, luminescent radiation in a first bandwidth and a second bandwidth that are optically isolatable from each other and that each have an intensity that varies as a known function of the luminescent material;
a source that exposes said quantity of luminescent material to an excitation energy, thereby causing said quantity of luminescent material to luminesce;
a photodetector system that detects luminescent radiation from the quantity of luminescent material in the first bandwidth and the second bandwidth thereby respectively generating a first electrical signal proportional to said first bandwidth and a second electrical signal proportional to said second bandwidth; and
a signal processor, responsive to the first electrical signal and the second electrical signal, that determines a temperature of the luminescent material and thus also a temperature of a tissue or an organ.

55. The system of claim 54, wherein said quantity of luminescent material comprises a composition (RE)₂O₂S:X, wherein
RE is an element selected from the group consisting of lanthanum, gadolinium and yttrium; and
X has a concentration of from 0.01 to 10.0 atom percent by weight and is selected from the group consisting of europium, terbium, praseodymium, samarium, dysprosium, holmium, erbium, thulium, neodymium and ytterbium.

56. The system of claim 54, wherein said quantity of luminescent material is positioned so that it is in thermal communication with a surface of a tissue so that a temperature of a tissue determined by said signal processor is a surface temperature of a tissue.

57. The system of claim 54, further comprising a catheter that contains said quantity of luminescent material and penetrates a surface of a tissue or an organ so that a temperature of a tissue or an organ determined by said signal processor is a sub-surface temperature of a tissue or an organ.

58. The system of claim 54, wherein said energy generator is an R-F generator, a laser generator, a microwave source, or an acoustic source.

59. The system of claim 54, wherein said source is radioactive material, a source of cathode rays, or an ultraviolet electromagnetic energy source.

60. A method, comprising:
applying energy to a tissue site or an organ site by an ablation electrode or a laser probe; and
monitoring a temperature of the tissue site or the organ site during said applying step.

61. The method of claim 60, wherein said monitoring comprises:
exposing a quantity of luminescent material to the tissue site or the organ site so that the quantity of luminescent material is responsive to a temperature of the tissue site or the organ site, said quantity of luminescent material being characterized by emitting, when excited with a transient radiation source, luminescent radiation in the visible spectrum having an intensity which decreases after termination of the transient radiation;
pulsing said quantity of luminescent material with an excitation radiation pulse, thereby causing said quantity of luminescent material to luminesce after termination of the pulse with a decreasing intensity function having a decay time that is related to the temperature of the quantity of luminescent material;
detecting a luminescent radiation of the quantity of luminescent material as it decreases in intensity thereby generating an electrical signal proportional thereto; and
measuring a decreasing characteristic of said electrical signal, thereby determining a quantity that corresponds to the temperature of the luminescent material and thus also to the temperature of the tissue site or the organ site.

62. The method of claim 61, wherein said quantity of luminescent material is positioned so that it is in thermal communication with a surface of the tissue site or the organ site and the temperature monitored during said monitoring step is a temperature of the surface of the tissue site or the organ site.

63. The method of claim 61, wherein said quantity of luminescent material is in a catheter that penetrates the tissue site or the organ site and the temperature monitored during said monitoring step is a sub-surface temperature of the tissue site or the organ site.

64. The method of claim 60, wherein said temperature of the tissue site or the organ site that is monitored during said applying step is a sub-surface temperature and wherein the monitoring comprises measuring an infrared electromagnetic energy emitted by a surface of the tissue site or the organ site with a pyrometer.

65. The method of claim 64, wherein said sub-surface temperature is a temperature of the tissue site or the organ site at least 1 mm below a surface of the tissue or the organ site.

66. The method of claim 64, wherein said sub-surface temperature is a temperature of the tissue site or the organ site at least 2 mm below a surface of the tissue or the organ site.

67. The method of claim 64, wherein said infrared electromagnetic energy is in a wavelength range between 0.9 microns and 1.9 microns.

68. The method of claim 64, the method further comprising calibrating said pyrometer.

69. The method of claim 68, wherein the calibrating comprises:
exposing a quantity of luminescent material to the tissue site or the organ site so that the quantity of luminescent material is responsive to a temperature of the tissue site or the organ site, said quantity of luminescent material being characterized by emitting, when excited with a transient radiation source, luminescent radiation in the visible spectrum having an intensity which decreases after termination of the transient radiation;
pulsing said quantity of luminescent material with an excitation radiation pulse, thereby causing said quantity of luminescent material to luminesce after termination of the pulse with a decreasing intensity function having a decay time that is related to a temperature of the quantity of luminescent material;
detecting a luminescent radiation of the quantity of luminescent material as it decreases in intensity thereby generating an electrical signal proportional thereto; and
measuring a decreasing characteristic of said electrical signal, thereby determining a quantity that corresponds to the temperature of the luminescent material.

70. The method claim 69, wherein said quantity of luminescent material is positioned so that it is in thermal communication with a surface of the tissue site or the organ site so that the temperature of the luminescent material is a surface temperature of the tissue site or the organ site.

71. The method of claim 69, wherein said quantity of luminescent material is positioned in a catheter that penetrates a surface of the tissue site or the organ site so that the temperature of the luminescent material is a sub-surface temperature of the tissue site or the organ site.

72. The method of claim 68, wherein the calibrating comprises:
exposing a quantity of luminescent material to the tissue site or the organ site so that the quantity of luminescent material is in thermal communication with the tissue site or the organ site, said quantity of luminescent material being characterized by emitting, when excited with a transient radiation source, luminescent radiation in a first bandwidth and a second bandwidth that are optically isolatable from each other and that each have an intensity that varies as a known function of the luminescent material;
applying a source of excitation energy to said quantity of luminescent material thereby causing said quantity of luminescent material to luminesce;
detecting luminescent radiation from the quantity of luminescent material in the first bandwidth and the second bandwidth thereby respectively generating a first electrical signal proportional to said first bandwidth and a second electrical signal proportional to said second bandwidth;
evaluating said first electrical signal and said second electrical signal to determine the temperature of the luminescent material and thus also to the temperature of the tissue site or the organ site thereby calibrating said pyrometer.

73. The method of claim 72, wherein said quantity of luminescent material comprises a composition (RE)₂O₂S:X, wherein
RE is an element selected from the group consisting of lanthanum, gadolinium and yttrium; and
X has a concentration of from 0.01 to 10.0 atom percent by weight and is selected from the group consisting of europium, terbium, praseodymium, samarium, dysprosium, holmium, erbium, thulium, neodymium and ytterbium.

74. The method of claim 72, wherein said quantity of luminescent material is positioned so that it is in thermal communication with a surface of the tissue site or the organ site so that the temperature of the tissue site or the organ site determined by said evaluating step is a surface temperature of the tissue site or the organ site.

75. The method of claim 72, wherein said quantity of luminescent material is in a catheter that penetrates a surface of the tissue site or the organ site so that a temperature of the tissue site or the organ site determined in said evaluating step is a sub-surface temperature of the tissue site or the organ site.

76. The method of claim 60, wherein said monitoring comprises:
exposing a quantity of luminescent material to the tissue site or the organ site so that the quantity of luminescent material is in thermal communication with the tissue site or the organ site, said quantity of luminescent material being characterized by emitting, when excited with a transient radiation source, luminescent radiation in a first bandwidth and a second bandwidth that are optically isolatable from each other and that each have an intensity that varies as a known function of the luminescent material;
applying a source of excitation energy to said quantity of luminescent material thereby causing said quantity of luminescent material to luminesce;
detecting luminescent radiation from the quantity of luminescent material in the first bandwidth and the second bandwidth thereby respectively generating a first electrical signal proportional to said first bandwidth and a second electrical signal proportional to said second bandwidth;
evaluating said first electrical signal and said second electrical signal to determine the temperature of the luminescent material and thus also to the temperature of the tissue site or the organ site.

77. The method of claim 76, wherein said quantity of luminescent material comprises a composition (RE)₂O₂S:X, wherein
RE is an element selected from the group consisting of lanthanum, gadolinium and yttrium; and
X has a concentration of from 0.01 to 10.0 atom percent by weight and is selected from the group consisting of europium, terbium, praseodymium, samarium, dysprosium, holmium, erbium, thulium, neodymium and ytterbium.

78. The method of claim 76, wherein said quantity of luminescent material is positioned so that it is in thermal communication with a surface of the tissue site or the organ site so that said temperature of the tissue site or the organ site determined by said evaluating step is a surface temperature of the tissue site or the organ site.

79. The method of claim 76, wherein said quantity of luminescent material is in a catheter that penetrates the tissue site or the organ site so that the temperature of the tissue site or the organ site determined in said evaluating step is a sub-surface temperature of the tissue site or the organ site.

80. The method of claim 60, wherein the tissue site or the organ site is a site of a tissue disease.

81. The method of claim 60, wherein said tissue disease is a liver anomaly, stomach cancer, bowel cancer, pancreatic cancer, kidney cancer, or lung cancer.

82. The method of claim 60, wherein the tissue site or organ site is ablated during the applying step to a controlled depth by plasma-induced volumetric removal of a tissue or a portion of an organ.

83. The method of claim 60, wherein the tissue site or the organ site is exposed to a temperature in the range of 40° C. to 90° C. during said applying step.

84. The method of claim 60, wherein the tissue site is skin.

85. The method of claim 60, wherein the organ site is a site on the heart, bladder, lung, liver, muscle, salivary gland, colon, spleen, pancreas, gallbladder, liver, kidney, stomach, tongue, thyroid gland, gallbladder, brain, large intestine, or small intestine.
